(19) United States
(12) Patent Application Publication
(10) Pub. No.: US 2024/0110187 A1
(43) Pub. Date: Apr. 4, 2024

(54) METHODS FOR PROPHYLACTIC AND THERAPEUTIC TREATMENT OF 2019-nCoV USING siRNAs AGAINST ORF1AB and N-Protein

(71) Applicant: Sirnaomics, Inc., Germantown, MD (US)

(72) Inventors: David M. EVANS, Germantown, MD (US); Wookhyun KIM, Germantown, MD (US)

(21) Appl. No.: 18/111,335

(22) Filed: Feb. 17, 2023

Related U.S. Application Data
(63) Continuation of application No. PCT/US22/49459, filed on Nov. 9, 2022.
(60) Provisional application No. 63/277,600, filed on Nov. 9, 2021.

(51) Int. Cl. C12N 15/113; A61K 31/713; A61P 31/14

(52) U.S. Cl. CPC C12N 15/1131; A61K 31/713; A61P 31/14; C12N 2310/14; C12N 2310/321; C12N 2310/322

(57) ABSTRACT

Methods are provided for prevention and treatment of 2019 coronavirus (2019-nCoV; COVID-19) infections in mammals by prophylactic or therapeutic administration of pharmaceutical compositions known as STP908. STP908 administered intravenously, whether prophylactically or therapeutically, led to survival of 50 percent of the treatment group infected with 2019-nCoV. STP908 compositions and methods of making them have been previously disclosed, and comprise potent siRNA therapeutics formulated in a histidine-lysine polymeric carrier; the siRNA molecules in STP908 target and reduce or inhibit the expression of two genes of the 2019-nCoV genome: ORF1AB and N-protein, preventing or ameliorating COVID-19 symptoms.

### CROSS REFERENCE TO RELATED APPLICATIONS

This application is a continuation of international application PCT/US2022/049459, filed Nov. 9, 2022, which claims the benefit of and priority to U.S. Provisional Patent Application No. 63/277,600, filed Nov. 9, 2021, the contents of which are incorporated herein by reference in their entirety.

### SEQUENCE LISTING

The instant application contains a sequence listing, submitted electronically in ST.26 (XML) format, is hereby incorporated by reference in its entirety. The ST.26 sequence listing, created on Feb. 7, 2023, is named 4690_0061I_SL and is 80 kilobytes in size.

### FIELD

Methods are provided for administration of nucleic acid-containing polymeric nanoparticles to prevent and treat 2019-nCoV infections and the resulting respiratory disease, COVID-19, in mammals. Administration of RNA-containing histidine-lysine polymeric nanoparticles modulate, interfere with, and/or inhibit genes that are a part of the 2019-nCoV genome, ORF1AB and N-protein.

### BACKGROUND

### 2019-nCoV Infection/COVID-19: Biology and Pathology

On Dec. 31, 2019, the World Health Organization (WHO) China Country Office was informed of cases of pneumonia of unknown etiology detected in Wuhan City, Hubei Province of China. The WHO reported that a novel coronavirus (2019-nCoV), a single-stranded, positive-sense RNA betacoronavirus (βCoV), was identified as the causative virus by Chinese authorities on 7 January (Lu H, Stratton C W, Tang Y W. Outbreak of Pneumonia of Unknown Etiology in Wuhan China: the Mystery and the Miracle. J Med Virol. 2020 January 16.). The 2019-nCoV is highly transmissible and the resulting respiratory disease, COVID19, has been fatal to large numbers of infected individuals. To-date over 250 million cases have been verified worldwide, and over 5 million people have died.

Symptoms of 2019-nCoV infection are similar to a range of other illnesses such as influenza, and include, among others, fever, coughing and difficulty breathing, the latter, which indicates the need for immediate medical attention. Clinical manifestations subsequent to infection include severe pneumonia, acute respiratory distress syndrome, septic shock and multi-organ failure. The 2019-nCoV infection may appear clinically milder than Severe Acute Respiratory Syndrome (SARS) or Middle East Respiratory Syndrome (MERS) in terms of fatalities and transmissibility, although new variants emerging from “super-spreader” evens may challenge this observation.

The 2019-nCoV is most closely related to two bat SARS-like coronavirus samples from China, initially suggesting that, like SARS and MERS, it may have originated in bats. (Ji W, Wang W, Zhao X, Zai J, Li X). Genomes and sub-genomes of coronaviruses (CoVs) contain at least 6 open reading frames (ORFs). The first ORF (ORF1a/b), about two-third of genome length, encodes 16 non-structural proteins (nsp1-16), except Gamma coronavirus that lacks nsp1. There is a −1 frameshift between ORF1a and ORF1b, leading to production of two polypeptides: pp1a and pp1ab. These polypeptides are processed by virally encoded chymotrypsin-like protease (3CLpro) or main protease (Mpro), and one or two papain-like protease (PLPs) into 16 nsps (Ziebuhr J, Snijder E J, Gorbalenya A E. Virus-encoded proteinases and proteolytic processing in the Nidovirales. J Gen Virol. 2000; 81 (Pt 4): 853-879.). Other ORFs on the one-third of genome near the 3′ terminus encode at least four main structural proteins: spike (S), membrane (M), envelope (E), and nucleocapsid (N) proteins.

A 2019-nCoV was isolated from a patient in January 2020 and subjected to genome sequencing, showing that the 2019-nCoV is a βCoV of group 2B with at least 70 percent similarity in genetic sequence to SARS-CoV. Sequence analysis showed that the 2019-nCoV possesses a typical genome structure of a CoV and belongs to the cluster of βCoVs that include Bat-SARS-like (SL)-ZC45, Bat-SL ZXC21, and SARS-CoV.

### RNAi

RNA interference (RNAi) is a sequence-specific RNA degradation process that provides a relatively easy and direct way to knockdown, or silence, theoretically any gene. In naturally occurring RNA interference, a double stranded RNA is cleaved by an endonuclease into small interfering RNA (siRNA) molecules, overhangs at the 3′ ends. These siRNA molecules are incorporated into a multicomponent-ribonuclease called RNA-induced-silencing-complex (RISC). One strand of siRNA remains associated with RISC and guides the complex towards a cognate RNA that has sequence complementary to the guider single stranded siRNA (ss-siRNA) in RISC. This siRNA-directed endonuclease digests the RNA, thereby inactivating it. Studies have revealed that the use of chemically synthesized 21-25-nucleotide (nt) siRNA molecules exhibit RNAi effects in mammalian cells, and the thermodynamic stability of siRNA hybridization (at or between terminals) plays a central role in determining the molecule's function.

### SUMMARY

Methods are provided for preventing and treating 2019-nCoV infections in mammals using IV or IT (intratracheal) administration of nucleic acid-containing pharmaceutical compositions. The compositions (described in detail in international publication WO2021/151096 which is hereby incorporated by reference in its entirety) comprise nucleic acids (e.g., siRNA, mRNA, and miRNA) and histidine-lysine copolymers carrier such as HKP or HKP(+H). When formulated, the components self-assemble as nanoparticles that, once inside the target cells, effectively modulate, interfere with, and/or inhibit sections (genes) of the viral genome, ORF1AB and N-protein gene expression. The compositions comprising siRNA molecules targeting the expression of ORF1AB and N-protein genes, also known as Simaomics' product STP908.

In certain embodiments methods are provided for preventing the 2019-nCoV infection by prophylactically administering a pharmaceutically effective amount of the siRNA molecules targeting the expression of ORF1AB and N-protein genes to a subject in need. In certain embodiments, administration may take place prior to exposure of the subject to 2019-nCoV. In a variety of embodiments, administration can take place from up to 2 weeks to within 3 to ˜24 hours of exposure to the virus.

In other embodiments methods are provided for treating a subject who has been infected by administering to the subject a pharmaceutical composition comprising the siRNA molecules targeting ORF1AB and N-protein genes, or to a subject suspected of being infected, with 2019-nCoV. In some embodiments, the subject has not exhibited any of the known symptoms of the infection. In a number of embodiments, the pharmaceutical composition is administered at each of a number of time periods following the subject's exposure to the virus: within at least 3 hours and up to 8 weeks following any suspected or known exposure to the virus.

In some embodiments methods are provided for slowing the progression of a 2019-nCoV infection in a subject, comprising administering to the subject a pharmaceutically effective amount of the pharmaceutical composition comprising the siRNA molecules targeting ORF1AB and N-protein genes. In certain embodiments, the pharmaceutical composition is administered within 3 hours and up to 8 weeks following exposure of the subject to the virus.

In all embodiments, the compositions may be administered through a variety of routes, including, but not limited to, intratracheal (IT), intranasal (inhaled), and intravenous (IV) injection or infusion. The subject is a mammal, in particular, a human, or alternatively a non-human primate, rat, mouse, ferret, or other mammal.

### BRIEF DESCRIPTION OF THE DRAWINGS

FIG. 1 depicts the PNP platform for delivering siRNA molecules, together with histidine-lysine copolymer in the same nanoparticle.

FIGS. 2A and 2B show survival data following prophylactic (2A) and therapeutic (2B) IP administration (to the lung) of two siRNA molecules against M2 and PA segments of the flu virus in mice infected with the Influenza virus A/California/07/09(H1N1)pmd09. The two siRNA molecules, formulated in a histidine-lysine copolymer (HKP), targeted the M2 and PA segments of the virus. The siRNA duo provided efficacy against H1N1.

FIG. 3 shows (left) the study design for evaluating mouse lung tissue viral load following prophylactic IV administration of the pharmaceutical composition comprising siRNA molecules targeting ORF1AB and N-protein, also know as Sirnaomics' product, STP908. Data graphed (right) indicate the effect of treatment to reduce the viral load in lung tissue in infected mice vs. infected, untreated controls; this also confirms that IV administration permits siRNAs to penetrate lung tissue of infected mice. The study is described in Example 1 below.

FIG. 4 shows a schematic of a study design for evaluating the efficacy of prophylactic or therapeutic STP908 administration in mice infected with 2019-nCoV. The study is described in Example 2 below.

FIG. 5 shows the rapid change (loss) of body weight due to infection, and, for STP908 delivered prophylactically through the IV route, a rebound in body weight. All mice in the IT-administered STP908 died, as did the controls by Day 7 or 8. Mice received one dose of STP908 prophylactically through intratracheal (IT) or intravenous (IV) administration routes, and three doses following the date of 2019-nCoV infection.

FIG. 6 (A-D) show the change in body weight progression for individual mice in the control (A-B) and treatment groups (C-D). None of the mice that received STP908 prophylactically through intratracheal (IT) administration maintained and gained weight; all died or were euthanized due to extreme weight loss by Day 8 (6C). For the mice administered STP908 intravenously (6D), three of the five animals initially lost weight as the infection progressed in the first week but recovered through the second week of the study.

FIG. 7 shows the change (loss) of body weight due to infection, and, for only the group of mice that received intravenous (IV) therapeutic administration of STP908, recovery from the infection through the second week of the study. Mice received therapeutic intratracheal (IT) or intravenous (IV) administration of STP908 (one dose) on the date of and (three doses) after infection with 2019-nCoV, but only IV administration of STP908 was efficacious.

FIGS. 8A-8D show the change in body weight progression for individual mice in the control (8A-B) and treatment groups (8C-D). All mice that received STP908 after infection with 2019-nCoV through intratracheal (IT) administration had died or were euthanized due to extreme weight loss by Day 6 or 7 (8C). For the mice administered STP908 intravenously (8D) following infection, 3 of the 6 recovered in the second week following infection.

FIG. 9 shows mortality in both the prophylactic and therapeutic STP908 regimens. All mice in the control groups had died or were euthanized by Day 7 or 8 post infection. Fifty (50) percent of mice were protected through both prophylactic and the therapeutic IV regimens, living to Day 14.

### DETAILED DESCRIPTION

Methods are provided for the prevention and treatment of 2019-nCoV infections by administering a pharmaceutical composition comprising two small interfering RNA (siRNA) molecules formulated with a histidine-lysine copolymer into nanoparticles. The two siRNAs packaged into each nanoparticle, when delivered to a target cell, are internalized and inhibit or reduce the expression of at least two genes of interest on the 2019-nCoV genome, ORF1AB and N-protein, known as Sirnaomics' product, STP908, reducing or inhibiting production of the virus and improving the health of the subject. The pharmaceutical compositions for involving siRNA products, comprising this, together with the histidine-lysine copolymer carrier, have been previously disclosed in detail in international publication WO2021/151096 (see supra) and published US application US 2021/0246448, including siRNA sequences identified below targeting the ORF1AB and N-Protein targets.

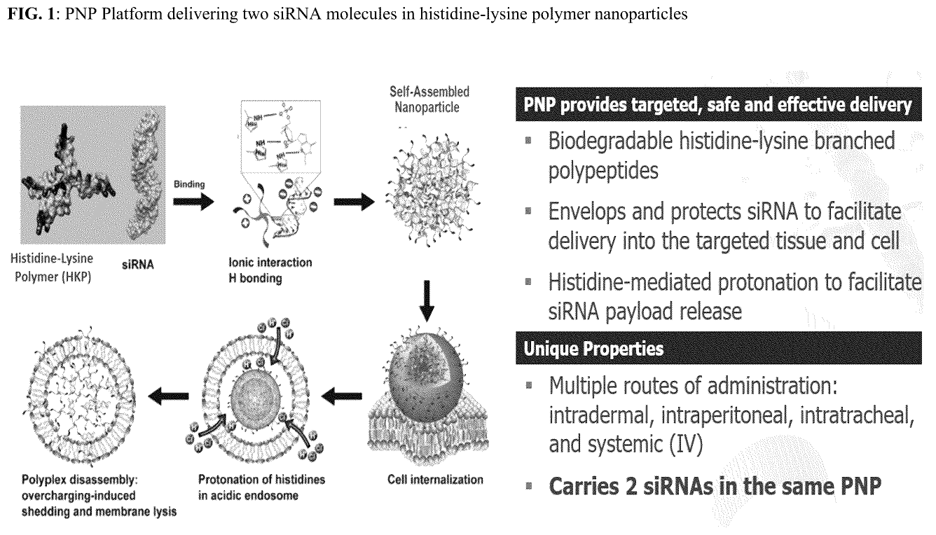

The PNP platform for delivering one or more nucleic acids (siRNA, miRNA, mRNA) molecules to targeted tissues and cells is shown in FIG. 1 and has been described previously. Briefly, products such as the combination siRNAs targeting ORF1AB and N-Protein genes are formulated as pharmaceutical compositions comprising nucleic acids (here, two siRNAs targeting the ORF1AB and N-protein genes of the 2019-nCoV) and histidine-lysine copolymers, which, when mixed as aqueous solutions in a 2.5:1 to 4:1 (HKP:siRNA) ratio, spontaneously form nanoparticles with an average diameter of 100-150 nm, capable of being internalized in targeted cells following local or systemic administration by any number of routes. The particles disassemble once inside the cells, releasing the nucleic acids to exert their respective effects. Further detail on histidine-lysine copolymer carriers has been discussed previously in U.S. Pat. No. 9,642,873, in international publication WO2021/151096 and elsewhere.

### ORF1AB and N-protein and Their Measurement

ORF1AB and N-protein expression can be measured by real time quantitative reverse transcription PCR (qRT-PCR) or other methods that are well known in the art.

### RNAi

RNA interference (RNAi) is a naturally occurring, highly specific mode of gene regulation. The mechanics of RNAi are both exquisite and highly discriminating. At the onset, short (19-25 bp) double-stranded RNA sequences (referred to as short interfering RNAs, siRNAs) associate with the cytoplasmically localized RNA Interference Silencing Complex (RISC). The resultant complex then searches messenger RNAs (mRNAs) for complementary sequences, i.e., target genes or sections thereof, eventually degrading (and/or attenuating translation of) these transcripts. Scientists have co-opted the endogenous RNAi machinery to advance a wide range of uses for siRNAs, including as therapeutics.

The nucleic acid may be a small interfering RNA (siRNA) molecule, comprising a double stranded (duplex) oligonucleotide, wherein the oligonucleotide targets a complementary nucleotide sequence in a single stranded (ss) target RNA molecule. The ss target RNA target molecule is an mRNA encoding at least part of a peptide or protein whose activity promotes inflammation, adipose tissue remodeling or sculpting, wound healing, or scar formation in skin tissue, or it is a micro RNA (miRNA) functioning as a regulatory molecule. siRNA sequences may be prepared in such way that each duplex can target and inhibit the same gene from, at least, both human and mouse, or non-human primates. In certain embodiments, an siRNA molecule binds to an mRNA molecule that encodes at least one protein with 100 percent or less complementarity. In further embodiments, an siRNA molecule binds to a mRNA molecule that encodes at least one human protein. In still additional embodiments, an siRNA molecule binds to a human mRNA molecule and to a homologous mouse mRNA molecule, i.e., mRNAs in the respective species that encode the same or similar protein.

“RNA” refers to a molecule comprising at least one, and preferably at least 4, 8 and 12 ribonucleotide residues. The at least 4, 8 or 12 RNA residues may be contiguous. By “ribonucleotide” is meant a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the dsRNA or internally, for example at one or more nucleotides of the RNA. Nucleotides in the RNA molecules of the disclosed embodiments can also comprise non-standard nucleotides, such as non-naturally occurring nucleotides or chemically synthesized nucleotides or deoxynucleotides. These altered RNAs can be referred to as analogs or analogs of naturally occurring RNA. As used herein, the term “siRNA” refers to a double stranded nucleic acid in which each strand comprises RNA, RNA analog(s) or RNA and DNA. Typically, the antisense strand of the siRNA is sufficiently complementary with the identified target sequences.

### ORF1AB and N-Protein siRNA Sequences

As identified in WO2021/151096 and US 2021/0246448, a larger number of siRNA sequences targeting ORF1AB and N-Protein were identified for use in combination in pharmaceutical compositions. The number of sequences was narrowed to develop the STP908 pharmaceutical composition product comprising SEQ ID Nos. 1-2 and 6-7. In some embodiments, the N-Protein-targeting siRNA sequences may be modified, as shown below in SEQ ID Nos. 3-5 and 8-10.

| SEQ ID | Sequence | Target | |
|---|---|---|---|
| NO: | Name | Gene | 5′ to 3′ Sequence |
| 1 and 6, | STP908-3 | ORF1AB | Sense: |
| respectively | | | GGAAGGAAGUUCUGUUGAAdTdT |
| | | | Antisense: |
| | | | UUCAACAGAACUUCCUUCCdTdT |
| 2 and 7, | STP908-18 | N-protein | Sense: |
| respectively | | | CCACCAACAGAGCCUAAAA-dTdT |
| | | | Antisense: |
| | | | UUUUAGGCUCUGUUGGUGG-dTdT |
| 3 and 8, | STP908- | N-protein | Sense: |
| respectively | 18mod1 | | CCACCAaCagaGcCuAaAa-dTdT |
| | | | Antisense: |
| | | | UuUUAgGCUCUgUuGgUgG-dTdT |
| 4 and 9, | STP908- | N-protein | Sense: |
| respectively | 18mod2 | | CCACCAACAGAGCCUAAAA-dTdT |
| | | | Antisense: |
| | | | UuUUAgGCUCUgUuGgUgG-dTdT |
| 5 and 10, | STP908- | N-protein | Sense: |
| respectively | 18mod3 | | CCACCAaCagaGcCuAaAa-dTdT |
| | | | Antisense: |
| | | | UuUuAgGgCuCuGuUgGuGg-dTdT |

Normal Capital letters are unmodified bases, Bold Capital letters are 2′OMe modified, Bold small case letters are 2′Fluoro modified.

The ORF1AB and N-Protein-targeting siRNAs in combination provides the benefit of avoiding the ability for the 2019-nCoV virus to escape therapeutic pressure unless it mutates in the regions of both the ORF1AB segment as well as the N-protein segment specifically targeted by the siRNA sequences. The two siRNA molecules are delivered using a nanoparticle formulation to ensure that they both are delivered to the same cell simultaneously.

### Prevention of and Treatment for 2019-nCoV Infection/COVID-19 and Other Viral Infections

Prophylactic and therapeutic methods are provided for preventing or treating a 2019-nCoV infection (and COVID-19) in subject who may or may not have been exposed to the virus or who has been diagnosed with the infection, with the goals of ameliorating symptoms, slowing the progression of the disease and, ultimately, of reducing the possibility of the subject's death in the most severe COVID-19 cases. Methods for preventing or treating such a viral infection comprise administering the pharmaceutical composition comprising siRNAs targeting ORF1AB and N-Protein genes through IV, IT, or other routes, as discussed below. The composition may be used in methods for the treatment of rapidly emerging influenza virus strains with high mortality rates that do not respond to existing therapies, while vaccines to protect the general population are under development. Further, the composition may provide significant value as a prophylactic/therapeutic with broad anti-influenza strain coverage and this coverage may well extend to as yet unidentified Influenza strains that may emerge in the future.

“Treatment,” or “treating” as used herein, is defined generally as the application or administration of a therapeutic agent (e.g., a dsRNA agent or vector or transgene encoding same) to a subject, or application or administration of a therapeutic agent to an isolated tissue or cell line from a subject, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

One aspect of the disclosed embodiments relates to treating subjects prophylactically, i.e., either to prevent the onset of symptoms of an infection, or to lessen the severity of symptoms if a 2019-nCoV infection should occur. In either case, the subject may not know whether s/he has been exposed to the virus at the time of pharmaceutical composition administration. In other embodiments, the subject may know that s/he was exposed to the virus.

Another aspect of the disclosed embodiments pertains to methods of treating subjects therapeutically once exposure or suspected exposure has taken place, i.e., altering the onset or the anticipated onset of symptoms, or ameliorating existing symptoms of the 2019-nCoV infection.

In other aspects, methods of treatment using the ORF1AB and N-Protein siRNA-containing pharmaceutical composition may include combining it with one or more other treatments to prevent and treat a 2019-nCoV infection, including addressing amelioration of particular symptoms. In certain of these embodiments, the other treatments involve administration of other nucleic acids (other siRNA, miRNA, mRNA, etc.) to comprise a treatment regimen comprising 3, 4, 5 or up to 10 or more individual nucleic acids targeting up to 10 or more different genes. In still other embodiments the other treatments involve administration of one or more small molecules or aptamers, antibody-based treatments, etc. In other method embodiments the pharmaceutical composition and other treatment(s) combinations may be administered to prevent or treat other viral infections as well as for 2019-nCoV infection or its variants. Some method embodiments may involve administration of the pharmaceutical composition and the one or more other treatments simultaneously. Other method embodiments may involve staggered administration of the pharmaceutical composition and the other treatment(s), and further may involve prophylactic as well as therapeutic administration of the pharmaceutical composition and the same for any of the other treatments if such indications are approved.

Other method embodiments for preventing a viral infection such as 2019-nCoV or its variants comprise administering the pharmaceutical composition over periods varying in length from weeks to months prior to and/or following exposure. In still other method embodiments, administration of the pharmaceutical composition at full or partial doses at regular or irregular intervals prior to or following dosing with other treatments may avoid or lessen the severity of symptoms of a viral infection, including 2019-nCoV and its variants.

### Evaluation of Lung Tissue Viral Load Following IV Administration of the Pharmaceutical Composition, STP908, in K18 hACE2 Mice

To evaluate the efficacy of STP908, the pharmaceutical composition targeting ORF1AB and N-Protein genes on lung tissue viral load, STP908 was administered intravenously to k18 hACE2 mice prophylactically at Days −5 & −2, infected them with the Italian strain of 2019-nCoV IV at Day 0, and then administered (IV) two more doses of STP908 at Days 1 & 3. FIG. 3 shows a reduction in the number of copies of the virus 10 genome in lung tissue in the STP908-treated group compared to that in the infected control group (receiving only vehicle solution). See further detail in Example 1 below.

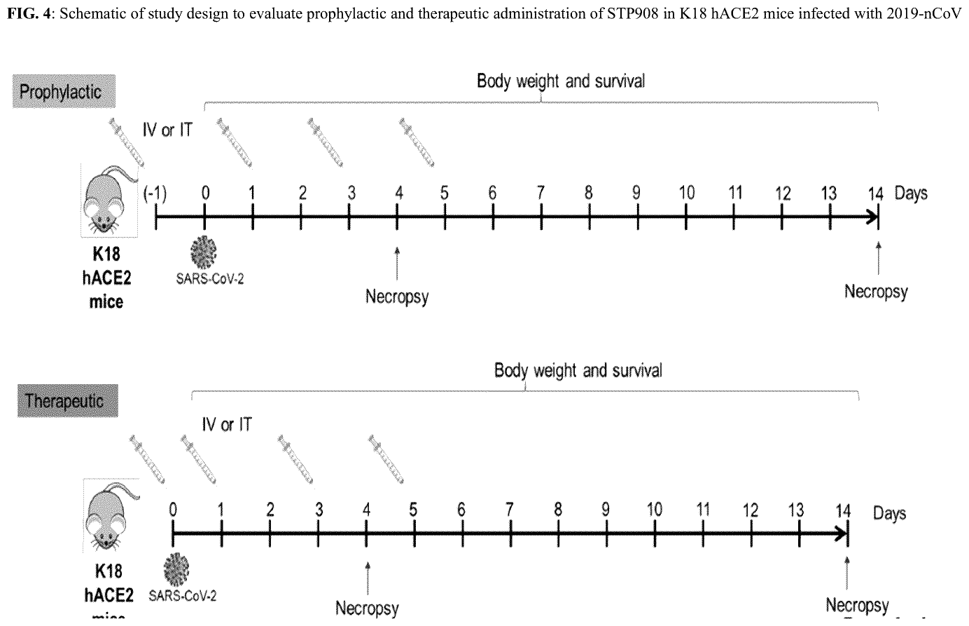

Timing and Mode of Administration in Mice. FIG. 4 shows a schematic of a study design for evaluating prophylactic and therapeutic administration of STP908 in k18 hACE2 mice infected with 2019-nCoV.

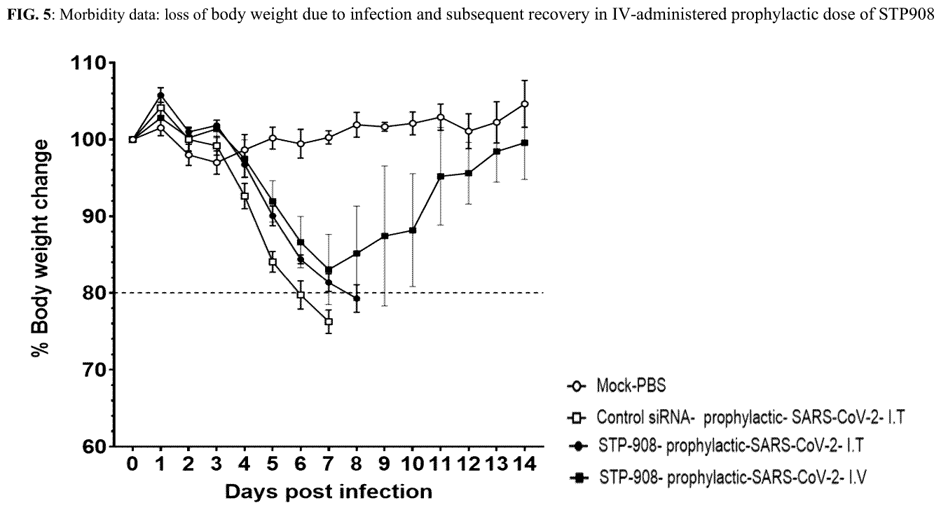

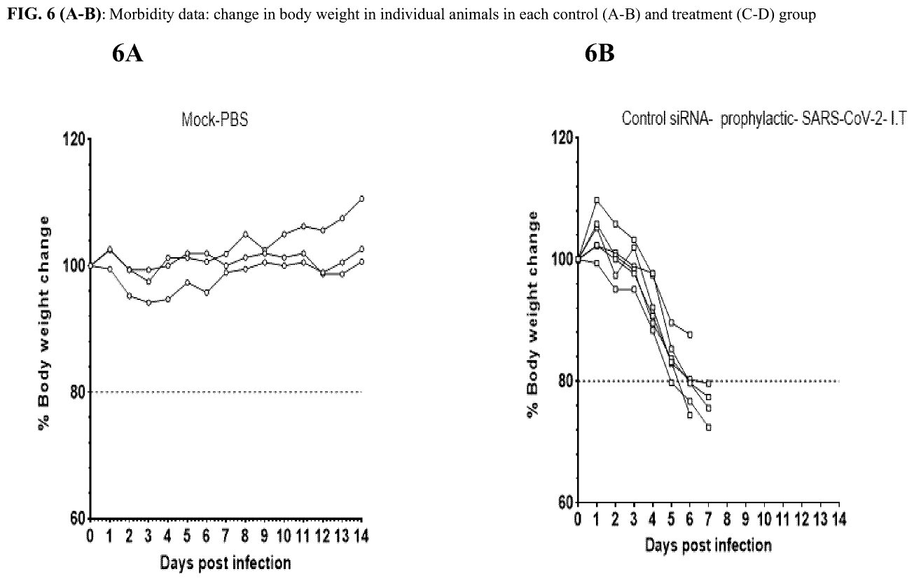

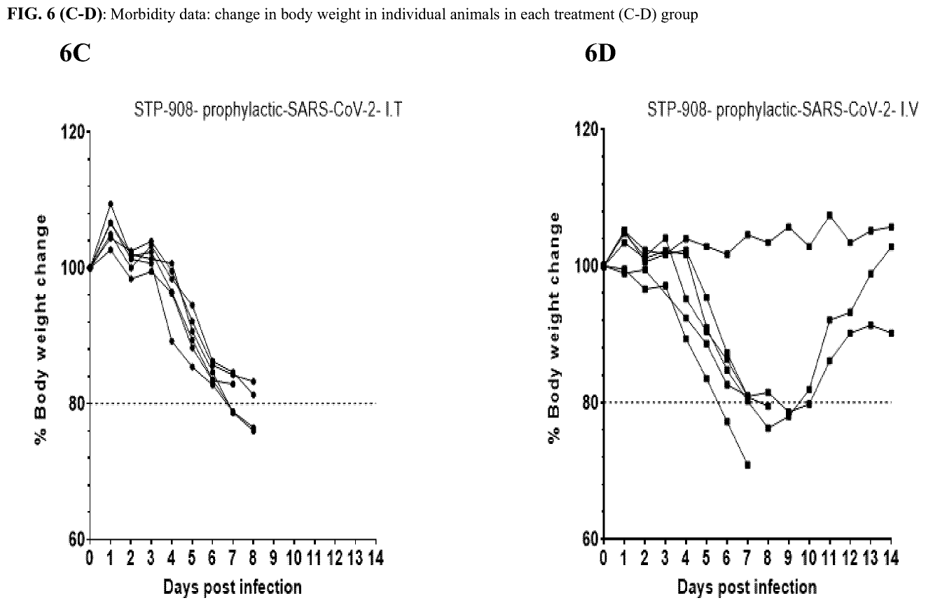

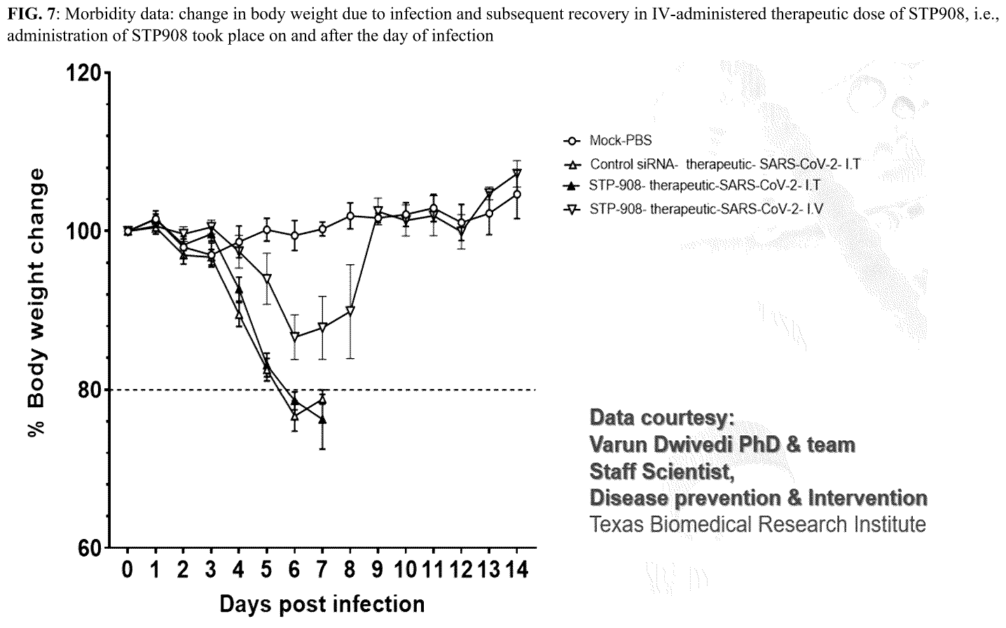

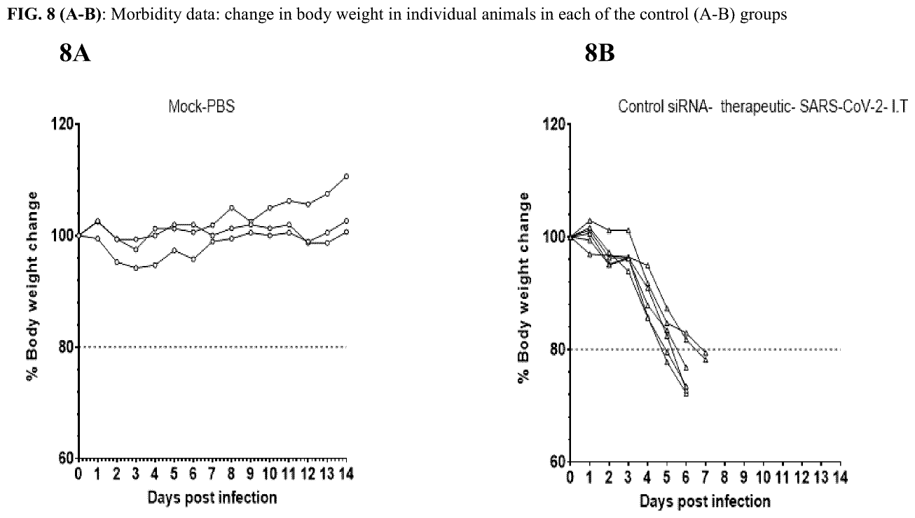

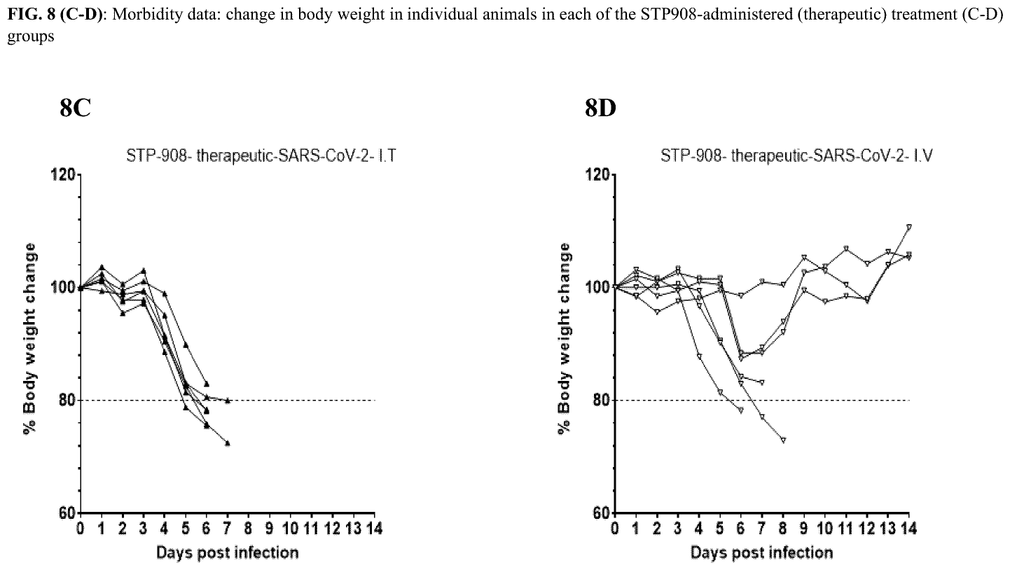

FIG. 5 (prophylactic administration, IT and IV) and FIG. 7 (therapeutic administration, IT and IV) show the rapid reduction in body weight within a few days after infection. The data show equally rapid recovery of body weight in the IV-administered treatment groups (IT and IV) when STP908 was administered prophylactically (FIG. 5) and therapeutically (FIG. 7). All mice in the IT-administered treatment groups died or had to be euthanized by Day 7 or 8. FIGS. 6A-6D and 8A-8D, depict the change of body weight post infection among individual mice in the four (2 prophylactic, 2 therapeutic) groups, showing an equally strong response to administration of STP908 among mice treated either prophylactically (6D) or therapeutically (8D) as long as administration was intravenous. Three of 6 mice in each IV-administered treatment group survived to Day 14. See further detail in Example 2 below.

### Dual siRNAs Targeting Influenza H1N1 Gene Expression to Ameliorate Symptoms of a Flu Infection

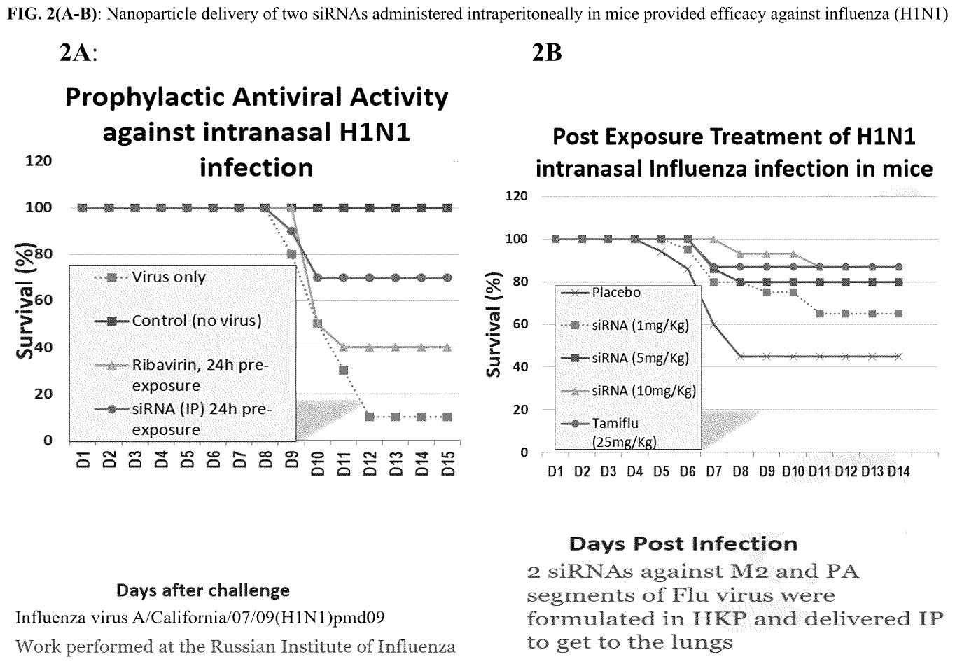

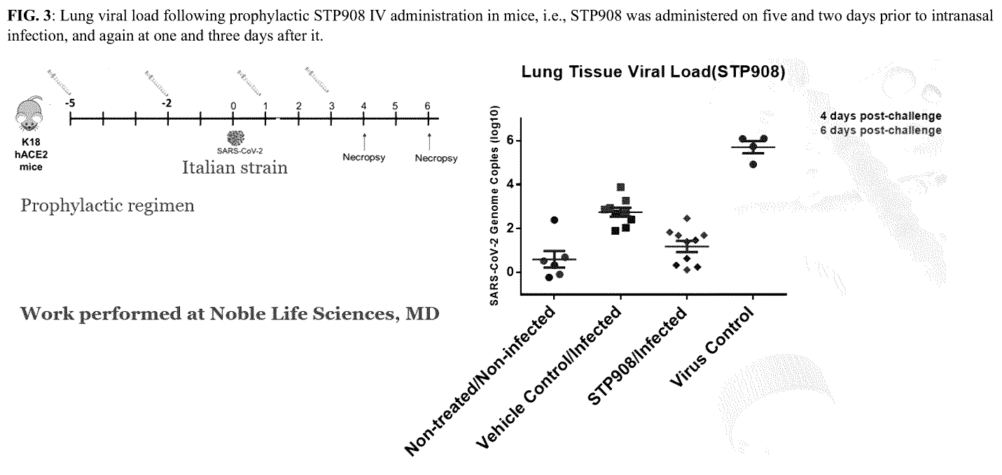

Influenza challenge in mice. The efficacy of using a pharmaceutical composition comprising two siRNA molecules formulated with a histidine-lysine copolymer to target genes on the mouse-adapted H1N1 virus strain has been described. See U.S. Pat. No. 9,868,952. Briefly, FIGS. 2A and 2B show survival data for following prophylactic (2A) and therapeutic (2B) IP administration (to the lung) of two siRNA molecules against the M2 and PA segments of the flu virus in mice infected with the Influenza virus A/California/07/09(H1N1)pmd09. We observed a dose-dependent inhibition of M2 and PA gene expression, and 50 percent of placebo-treated mice died by Day 8 following exposure to the virus. The prophylactic effect of the two siRNAs specific to H1N1 influenza genes exceeded the effect of Ribavirin. Further, administration of 10 mg/kg of the combination of siRNAs 24 hours post exposure to the virus resulted in more than 80 percent survival after Day 11, greater or somewhat similar to the effect seen using 25 mg/kg of Tamiflu (oseltamivir phosphate, Roche, Switzerland). Mice were also challenged with the same mouse-adapted H1N1 strain at 24, 48, 72 and 96 hours post challenge, which revealed that therapeutic intervention against H1N1 should begin within or by 24 hours following exposure to the virus.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype”, or “drug response genotype”). Thus, another aspect of the disclosed embodiments provides methods for tailoring a subject's prophylactic or therapeutic treatment with either the target ORF1AB or N-protein gene or modulator according to that subject's drug response genotype. Pharmacogenomics allows a clinician or physician to target prophylactic or therapeutic treatments to patients who will most benefit from the treatment and to avoid treatment of patients who will experience toxic drug-related side effects.

### Dosing

As previously disclosed, STP908, the pharmaceutical composition comprising siRNAs targeting ORF1AB and N-Protein genes can be formulated to comprise a pharmacologically effective amount of therapeutic agent, i.e., of each of the two siRNA molecules, along with a pharmaceutically acceptable carrier.

A pharmacologically or therapeutically effective amount refers to that amount of the RNA effective to produce the intended pharmacological, therapeutic or preventive result. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20 percent reduction in a measurable parameter associated with a disease or disorder, a therapeutically effective amount of a drug for the treatment of that disease or disorder is the amount necessary to effect at least a 20 percent reduction in that parameter.

As defined herein, a therapeutically effective amount of a nucleic acid molecule (i.e., an effective dosage) depends on the nucleic acid selected. For instance, single dose amounts of an RNA molecule (or, e.g., a construct(s) encoding for such RNA) in the range of approximately 1 pg to up to 10 mg may be administered; in some embodiments, 1, 10, 30, 100, or 1000 pg, or 10, 30, 100, or 1000 ng, or 10, 30, 100, or 1000 pg, may be administered in several areas of the body of a 60 to 120 kg subject (i.e., 0.1 μg/Kg to 2000 μg/Kg). In some embodiments, doses ranging from 60 to 150 μg are administered in this way. The compositions can be administered from one or more times per day to one or more times per week for the desired length of the treatment; including once every other day. The skilled artisan will appreciate that certain factors may influence the dosage and timing required to effectively treat a subject, including but not limited to the severity of the disease or disorder, previous treatments, the general health and/or age of the subject, and other diseases present. Treatment of a subject with a therapeutically effective amount of a nucleic acid (e.g., dsRNA), protein, polypeptide, or antibody can include a single treatment or, preferably, can include a series of treatments.

A pharmaceutical composition comprising the RNA can be administered once daily. However, the therapeutic agent may also be dosed in units containing two, three, four, five, six or more sub-doses administered at appropriate intervals throughout the day. In that case, the RNA contained in each sub-dose must be correspondingly smaller to achieve the total daily dosage unit. The dosage unit can also be compounded for a single dose over several days, e.g., using a conventional sustained release formulation which provides sustained and consistent release of the RNA over several days. Sustained release formulations are well known in the art. In this embodiment, the dosage unit contains a corresponding multiple of the daily dose. Regardless of the formulation, the pharmaceutical composition must contain RNA in a quantity sufficient to inhibit expression of the target gene in the animal or human being treated. The composition can be compounded in such a way that the sum of the multiple units of RNA together contain a sufficient dose.

Depending on the particular target gene sequence and the dose of RNA agent material delivered, this process may provide partial or complete loss of function for the target gene. A reduction or loss of expression (either target gene expression or encoded polypeptide expression) in at least 50%, 60%, 70%, 80%, 90%, 95% or 99% or more of targeted cells is exemplary. Inhibition of target gene levels or expression refers to the absence (or observable decrease) in the level of target gene or target gene-encoded protein. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target gene sequence(s) by the dsRNA agents of the disclosed embodiments also can be measured based upon the effect of administration of such dsRNA agents upon development/progression of a target gene associated disease or disorder, e.g., deleterious adipose tissue remodeling due to obesity, over feeding or a metabolic derangement, tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 105-fold, 106-fold, 107-fold reduction in cancer cell levels could be achieved via administration of the dsRNA agents of the disclosed embodiments to cells, a tissue, or a subject.

The data obtained from the cell culture assays and animal studies (toxicity, therapeutic efficacy) can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the method of the disclosed embodiments, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography (HPLC).

### Administration for Prevention and Treatment

Biological barriers protect the lungs from foreign particles. Examples include: (i) a thick mucus layer that may bind inhaled drugs and remove them via a mucus clearance mechanism, (ii) low basal and stimulated rates of endocytosis on the apical surfaces of well-differentiated airway epithelial cells, (iii) the presence of RNase extra- and intracellularly, and (iv) the presence of endosomal degradation systems in the target cells, among others. Overcoming the difficulties of respiratory tract delivery and effective cellular entry and function will pave the way for siRNA as a systemic anti-2019-nCoV therapeutic.

The delivery vehicle and mode of administration allow a rapid onset of gene silencing at the targeted site of action, e.g., early stage infection/prophylaxis at the epithelial/endothelial cells, and in later stage disease through systemic administration. Among them, topical/systemic delivery through inhalation has been shown an effective way to treat the respiratory system diseases. The data described herein demonstrate that systemic (IV) administration may, in some situations, provide a superior result when IT administration shows lower or even no efficacy. In a pandemic setting, either IT or IV administration may permit ease of administration directly to subjects.

Suitably formulated compositions of the disclosed embodiments can be administered by means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

COVID-19 is an infectious disease of the respiratory system. It enters the cells through the ACE2 receptors or Neuropilin 1 receptors on the epidermal cells of the respiratory tract and lungs to start the replication life cycle. Therefore, administration through the respiratory tract is a suitable effective mode of administration of STP908 in some situations. The pharmaceutical composition, STP908, may be delivered to the respiratory system, especially the lower respiratory tract and lungs through a specific inhalation device, which could effectively reach the focus of virus infection and replication to achieve high-efficiency of the inhibition of virus. One administration mode for the prevention and treatment of novel coronavirus infection is atomized inhalation administration. Specifically, a hand-held atomization device is used to atomize the nanoparticles preparation. The inhaled droplets of the drug preparation are atomized through the respiratory tract to deliver the drugs to the lower respiratory tract and lungs. The device preferably uses an ultrasonic atomization device, and more preferably, a micro-net ultrasonic atomization inhalation device.

A formulation is prepared to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The siRNA formulations can also be administered by transfection or infection using methods known in the art, including but not limited to the methods described in McCaffrey et al. (2002), Nature, 418 (6893), 38-9 (hydrodynamic transfection); Xia et al. (2002), Nature Biotechnol., 20 (10), 1006-10 (viral-mediated delivery); or Putnam (1996), Am. J. Health Syst. Pharm. 53 (2), 151-160, erratum at Am. J. Health Syst. Pharm. 53 (3), 325 (1996).

Further, the siRNA formulations can also be administered by a method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88 (2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

2019-nCoV infections or suspected 2019-nCoV infections may be treated to slow progression or infections may be prevented altogether by administering to a subject in need a pharmaceutically effective amount of the pharmaceutical composition comprising a combination of two siRNAs, one targeting the ORF1AB gene and the other targeting the N-protein gene, where the first siRNA targeting the ORF1AB gene is SEQ ID No. 1 and 6, and the second siRNA targeting the N-Protein gene is selected from the group consisting of SEQ ID Nos. 1-2 and 6-7, SEQ ID Nos. 1, 3, 6, and 8, SEQ ID Nos. 1, 4, 6, and 9, and SEQ ID Nos. 1, 5-6, and 10. This pharmaceutical composition may be administered to a subject initially within 3 hours, 6 hours, 12 hours, 18 hours, 1 day, 2 days, 3 days, 4 days, 5 days, 6 days, 8 days, 10 days, 2 weeks, 3 weeks, 4 weeks, 6 weeks or 8 weeks following exposure, suspected exposure or as a preventative measure prior to exposure of the subject to 2019-nCoV, regardless of whether the subject has exhibited any signs or symptoms of a 2019-nCoV infection.

In some embodiments the pharmaceutical composition comprises an siRNA combination of SEQ ID Nos. 1-2 and 6-7.

The following examples illustrate certain aspects of the disclosure and should not be construed as limiting the scope thereof.

### EXAMPLES

### Example 1

Evaluation of Lung Viral Load in k18 hACE2 Mice Following (i) Prophylactic IV Administration of STP908, (ii) Intranasal Infection with 2019-nCoV, and (iii) Further IV Administration of STP908

Methods: Thirty-two k18 hACE2 mice were assigned to four groups of eight each: (i) non-treated/non-infected controls; (ii) infected, vehicle-administered controls, (iii) virus controls (no treatment); and (iv) infected, STP908-administered treatment groups. At Days −5 and −2, the mice in group (iv) were prophylactically administered two doses of STP908, while the infected controls received only the vehicle solution. At Day 0 all but the group (i) non-treated, non-infected mice were intranasally infected with the Italian strain of the 2019-nCoV, and at Days 1 and 3 the mice in group (iv) again received two IV doses of STP908. At day 4, four mice in each group were euthanized and lung tissue samples were taken during necropsy to determine the number of copies of the 2019-nCoV genome in that tissue. At Day 6, the remaining four mice in each group were euthanized and tissue samples again were taken for analysis. FIG. 3 (on the left) shows a schematic of the study design.

Results: FIG. 3 (on the right) shows a reduction in the number of copies of the 2019-nCoV genome found in the lung tissue samples of mice that received IV-administered STP908 compared to infected control mice that received only the vehicle solution. The results confirmed that, within only a few days of prophylactic IV administration of STP908, siRNA molecules penetrated the lung tissue and, at least in part, inhibited expression of the two key genes targeted in the viral genome; as a result, replication of the viral genome was slowed or halted.

### Example 2

Evaluation of the Effect of Prophylactic and Therapeutic STP908 Administration via Intravenous (IV) and Intratracheal (IT) Routes k18 hACE2 Mice Infected with 2019-nCoV

Methods: Four to 6-week old k18 hACE2 female mice were randomized to 6 groups consisting of two control groups (PBS, mock infected, n=3; non-silencing (NS) siRNA (negative controls), n=10), and four treatments groups of 10 mice each receiving STP908 (2.5 mg/kg):
1. prophylactic IV administration of 40 μg on Day −1 & Days 1, 3, & 5 post infection;
2. prophylactic IT administration of 40 μg as with IV administration;
3. therapeutic IV administration of 40 μg on Days 0 (post infection) & Days 1, 3 & 5 post infection; and
4. therapeutic IT administration of 40 μg, as with IV administration.
   All but the PBS control group were inoculated intranasally with 1×10³ PFU/animal of the 2019-nCoV on Day 0. Four animals from each of the NS siRNA control and each treatment group were euthanized on Day 4 post infection and the following tissues were collected for analysis: (i) right caudal lung lobe (for histopathology if required); and (ii) remaining lung lobes and nasal turbinate (for homogenate preparation and determination of the viral load by plaque assay); the lung and nasal turbinate homogenate was also stored for cytokine determination (if required). All three PBS control group mice and the remaining 6 mice from each treatment group were monitored for morbidity and mortality until Day 14 when they were euthanized for the same tissue collection and analysis as indicated above. Any mice exhibiting labored breathing, eye discharge, nasal discharge, hunched posture and or substantial (20-25%) body weight loss were euthanized under IACUC instructions. Mice infected with the 2019-nCoV showed ˜60 percent survival by Day 14 post infection. FIG. 4 provides a schematic of the study design.

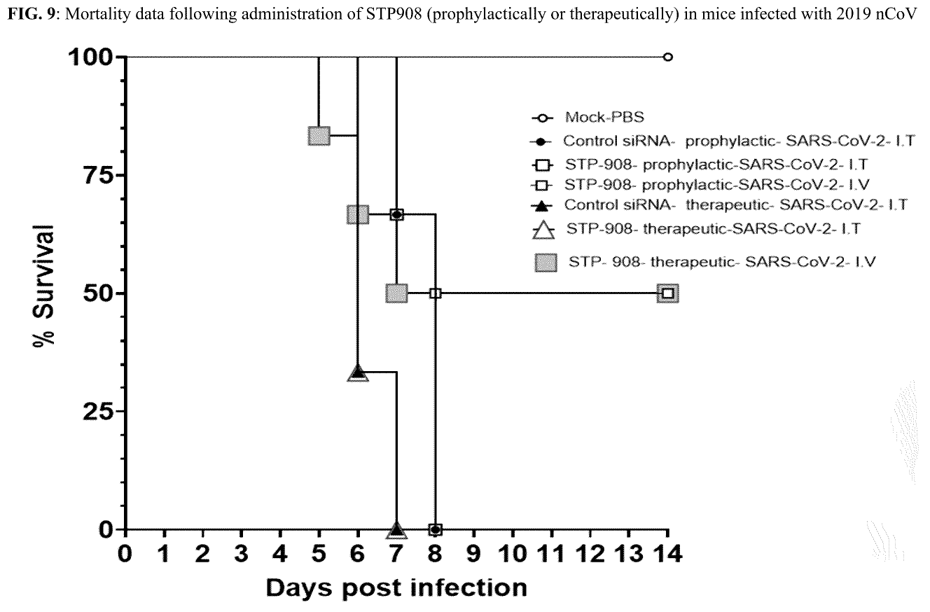

Results: FIGS. 5-9 provide morbidity and mortality data. FIG. 5 shows the initial loss and then gain of body weight among mice prophylactically administered STP908 through IV and IT routes, indicating that only IV administration was efficacious as early as 7 to 10 days post infection. As indicated above, as mice in the control siRNA group continued to lose a significant amount of weight they were euthanized per the IACUC rules. FIGS. 6A-6D depict the change of body weight post infection among individual mice in the four groups. While the body weights of 3 of the 5 of the mice in the IV administered group rebounded and the mice lived to Day 14 (6D), none of the mice in the IT administered group lived to Day 14 (6C), indicating some level of efficacy of STP908 through prophylactic IV administration. FIG. 7 again demonstrates the effectiveness of IV over IT administration when dosing takes place following infection. FIG. 7 shows the initial loss and then gain of body weight among mice therapeutically administered STP908 intravenously. All mice therapeutically administered STP908 intratracheally died by Day 7 or 8. As in FIGS. 6A-6D, FIGS. 8A-8D depict the change of body weight post infection among individual mice in the four groups. As shown in FIGS. 8C and 8D, 3 of 6 mice survived beyond Day 8, indicating that therapeutic IV—but not IT—administration of STP908, is efficacious in this animal model. Finally, FIG. 9 shows mortality data for all control and treatment groups. All mice in the control groups as well as all those administered STP908 through the IT route had died by Day 8 post infection; in contrast, both prophylactic and therapeutic IV administration of STP908 resulted in 50 percent survival of the STP908-treated groups at Day 14.

The disclosed embodiments described and claimed are not to be limited in scope by the specific preferred embodiments referenced herein, since these embodiments are intended as illustrations, not limitations. Any equivalent embodiments are intended to be within the scope of this disclosure, and the embodiments disclosed are not mutually exclusive. Indeed, various modifications to the embodiments, in addition to those shown and described herein will become apparent to those skilled in the art from the foregoing description. Such modifications are also intended to fall within the scope of the appended claims.

The terms and words used in the following description and claims are not limited to conventional definitions but, rather, are used to enable a clear and consistent understanding of the disclosure. Accordingly, it should be apparent to those skilled in the art that the description of various embodiments is provided for illustration purpose only and not for the purpose of limiting the disclosure with respect to the appended claims and their equivalents.

It is to be understood that the singular forms “a,” “an,” and “the” include the plural forms unless the context clearly dictates otherwise, e.g., reference to “a dermatologically active compound ” includes reference to one or more such compounds.

Unless otherwise defined herein, all terms used have the same meaning as commonly understood by a person of ordinary skill in the art. Terms used herein should be interpreted as having meanings consistent with their meanings in the context of the relevant art.

As used herein, the terms “comprising,” “comprise” or “comprised,” in reference to defined or described elements of any item, composition, formulation, apparatus, method, process, system, etc., are intended to be inclusive or open ended, and includes those specified elements or their equivalents. Other elements can be included and still fall within the scope or definition of the defined item, composition, etc.

The term “about” or “approximately” means within an acceptable error range for the particular value as viewed by one of ordinary skill in the art; this depends in part on how the value is measured or determined based on the limitations of the measurement system.

“Co-administer” or “co-deliver” refers to the simultaneous administration of two pharmaceutical formulations in the blood or other fluid of an individual using the same or different modes of administration. Pharmaceutical formulations can be concurrently or sequentially administered in the same pharmaceutical carrier or in different ones.

The terms “subject,” “patient,” and “individual” are used interchangeably.

What is claimed is:

1. A method of preventing and/or ameliorating a 2019-nCoV infection comprising administering to a subject in need thereof a pharmaceutically effective amount of a pharmaceutical composition comprising a first siRNA targeting ORF1AB and a second siRNA targeting N-protein, wherein said first siRNA is SEQ ID Nos. 1 and 6, and wherein said second siRNA is selected from the group consisting of SEQ ID Nos. 2-4 and 7-9 and SEQ ID Nos. 5 and 10.

2. The method of claim 1, wherein the pharmaceutical composition comprises an siRNA combination of SEQ ID Nos. 1-2 and 6-7.

3. The method of claim 1, wherein the pharmaceutical composition is administered to said subject prior to exposure of the subject to 2019-nCoV.

4. The method of claims 1, wherein said administration of the pharmaceutical composition to a subject takes place at least 2 weeks, 10 days, 8 days, 6 days, 5 days, 4 days, 3 days, 2 days 1 day, 18 hours, 12 hours, 6 hours or 3 hours prior to exposure of the subject to the 2019-nCoV.

5. The method of claim 1, wherein said pharmaceutical composition is administered intravenously.

6. The method of claim 1, wherein said pharmaceutical composition is administered intratracheally.

7. A method of treating a subject suffering from, or suspected of suffering from, 2019-nCoV infection, comprising administering to the subject a pharmaceutically effective amount of a pharmaceutical composition comprising a first siRNA targeting ORF1AB and a second siRNA targeting N-protein, wherein said first siRNA is SEQ ID Nos. 1 and 6, and wherein said second siRNA is selected from the group consisting of SEQ ID Nos. 2-4 and 7-9 and SEQ ID Nos. 5 and 10.

8. The method of claim 7, wherein the pharmaceutical composition comprises an siRNA combination of SEQ ID Nos. 1-2 and 6-7.

9. The method of claim 7, wherein said subject has not exhibited any known symptom of a 2019-nCoV infection.

10. The method of claim 7, wherein said pharmaceutical composition is administered at least 3 hours, 6 hours, 12 hours, 18 hours, 1 day, 2 days, 4 days, 6 days, 8 days, 10 days, 2 weeks, 3 weeks, 4 weeks, 6 weeks or 8 weeks following exposure of the subject to the 2019-nCoV.

11. The method of claim 7, wherein said pharmaceutical composition is administered intravenously.

12. The method of claim 7, wherein said pharmaceutical composition is administered intratracheally.

13. A method of slowing the progression of a 2019-nCoV infection in a subject comprising administering to the subject a pharmaceutically effective amount of a pharmaceutical composition comprising a first siRNA targeting ORF1AB and a second siRNA targeting N-protein, wherein said first siRNA is SEQ ID Nos. 1 and 6, and wherein said second siRNA is selected from the group consisting of SEQ ID Nos. 2-4 and 7-9 and SEQ ID Nos. 5 and 10.

14. The method of claim 13, wherein the pharmaceutical composition comprises an siRNA combination of SEQ ID Nos. 1-2 and 6-7.

15. The method of claim 13, wherein said pharmaceutical composition is administered within 3 hours, 6 hours, 12 hours, 18 hours, 1 day, 2 days, 4 days, 6 days, 8 days, 10 days, 2 weeks, 3 weeks, 4 weeks, 6 weeks or 8 weeks following exposure of the subject to the 2019-nCoV.

16. The method of claim 15, wherein said pharmaceutical composition is administered intravenously.

17. The method of claim 15, wherein said pharmaceutical composition is administered intratracheally.

18. The method of claim 1, wherein the subject is a mammal.

19. The method of claim 18, wherein the subject is selected from the group consisting of humans, non-human primates, mice, rats and ferrets.

20. The method of claim 18 wherein the subject is a human.


SEQUENCE LISTING (10 sequences)

SEQ ID NO: 1 - DNA, 21 bp, synthetic construct
  source 1..21: mol_type = other DNA
  misc_feature 1..19: note = RNA
  misc_feature 20..21: note = DNA
ggaaggaagttctgttgaatt

SEQ ID NO: 2 - DNA, 21 bp, synthetic construct
  source 1..21: mol_type = other DNA
  misc_feature 1..19: note = RNA
  misc_feature 20..21: note = DNA
ccaccaacagagcctaaaatt

SEQ ID NO: 3 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  misc_feature 1..19: note = RNA
  misc_feature 20..21: note = DNA
ccaccaacagagcctaaaatt

SEQ ID NO: 4 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
ccaccaacagagcctaaaatt

SEQ ID NO: 5 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  misc_feature 1..19: note = RNA
  misc_feature 20..21: note = DNA
ccaccaacagagcctaaaatt

SEQ ID NO: 6 - DNA, 21 bp, synthetic construct
  source 1..21: mol_type = other DNA
  misc_feature 1..19: note = RNA
  misc_feature 20..21: note = DNA
ttcaacagaacttccttcctt

SEQ ID NO: 7 - DNA, 21 bp, synthetic construct
  source 1..21: mol_type = other DNA
  misc_feature 1..19: note = RNA
  misc_feature 20..21: note = DNA
ttttaggctctgttggtggtt

SEQ ID NO: 8 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
ttttaggctctgttggtggtt

SEQ ID NO: 9 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
ttttaggctctgttggtggtt

SEQ ID NO: 10 - DNA, 22 bp, synthetic construct
  source 1..22: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide
  misc_feature 1..20: note = RNA
  misc_feature 21..22: note = DNA
ttttagggctctgttggtggtt